(19) United States
(12) Patent Application Publication
(10) Pub. No.: US 2017/0121645 A1
(43) Pub. Date: May 4, 2017

(54) DETERGENT COMPOSITION WITH SILICATE COATED BLEACH

(71) Applicant: The Procter & Gamble Company, Cincinnati, OH (US)

(72) Inventors: Philip Frank SOUTER, Northumberland (GB); Michelle JACKSON, Tyne & Wear (GB); Euan John MAGENNIS, Newcastle upon Tyne (GB)

(21) Appl. No.: 15/403,200

(22) Filed: Jan. 11, 2017

Related U.S. Application Data
(63) Continuation of application No. 15/076,980, filed on Mar. 22, 2016.
Application No. 15/076,980 is a continuation of application No. 14/132,054, filed on Dec. 18, 2013, now abandoned.
(60) Provisional application No. 61/834,939, filed on Jun. 14, 2013.
(60) Provisional application No. 61/739,796, filed on Dec. 20, 2012.

(51) Int. Cl. C11D 3/386; C11D 17/00; C11D 3/33; C12N 9/26; C11D 3/08; C11D 3/37; C12N 9/54; C11D 3/39; C11D 17/04

(52) U.S. Cl. CPC C11D 3/386; C11D 3/3942; C11D 17/0039; C11D 3/33; C11D 17/045; C11D 17/042; C11D 3/08; C11D 3/37; C12N 9/54; C12N 9/2411

(57) ABSTRACT

A phosphate-free automatic dishwashing detergent composition containing a coated bleach particle and one or more of an enzyme particle or an amino-acid based builder, wherein the composition has increased stability.

### TECHNICAL FIELD

The present invention is in the field of automatic dishwashing detergent. More specifically, the invention is in the field of phosphate free automatic dishwashing detergent compositions comprising stabilized bleach and one or more of enzyme particles and amino acid based builders.

### BACKGROUND OF THE INVENTION

Traditionally, phosphate builders have been used in detergent formulations. Environmental considerations have made it desirable to replace phosphate with more environmentally friendly builders. However, the replacement of phosphate builders can impair the stability of detergents. Phosphate contributes to the moisture management and stability of detergents by acting as a moisture sink. The majority of builders that can be used as replacements for phosphate are incapable of acting as a moisture sink, therefore contributing to the instability and degradation of the detergent over time. This drawback has a greater impact in detergents which comprise moisture sensitive ingredients such as bleach and enzymes.

Therefore, there is a need for phosphate free detergent compositions that have improved stability. In addition, there is a need for phosphate free detergent compositions that provide improved stability of detergent actives such as bleaches, amino-acid based builders, and enzymes.

### SUMMARY OF THE INVENTION

A phosphate-free automatic dishwashing detergent composition comprising: (a) a coated bleach particle comprising at least two layers, where in the coated bleach particle comprises: (i) a core substantially consisting of bleach; and (ii) an inner layer at least partially enclosing the core, wherein the inner layer comprises an efflorescent material; and (iii) an outer layer at least partially enclosing the inner layer, wherein the outer layer comprises a water-insoluble material; and one or more of: b)an enzyme particle comprising an efflorescent material and an enzyme, wherein the enzyme is selected from the group consisting of amylase, protease, and mixtures thereof; and c) an amino acid-based builder selected from the group consisting of methyl-glycine-diacetic acid and salts, glutamic-N,N-diacetic acid and salts, and mixtures thereof; and wherein the composition is substantially free of anionic and cationic surfactants.

### DETAILED DESCRIPTION

Enzymes are usually present in detergent compositions in granulate form with efflorescent material being added to the enzyme granulates in order to provide enzyme stability. By “efflorescent material” it is herein understood to mean a material that in its anhydrous form can take water to become hydrated and it can easily give up the hydration water when it is placed in a drier or warmer environment. The efflorescent materials for use in the composition of the invention have a difference in density between the anhydrous and hydrated form of at least 0.8 g/cm³, in another embodiment at least 1 g/cm³, and in another embodiment at least 1.2 g/cm³. This difference in densities provides a mechanism to break particle to particle crystal bridges that form as a result of water condensing as the powder temperature falls below the dew point associated with the powder. As the temperature increases following a period of cooling (as in a temperature cycle), the hydrated material forming a crystal bridge between particles reverts to the anhydrous (or less hydrated) form. The higher crystal density associated with the anhydrous (or less hydrated) form provides a mechanism for breaking these crystal bridges due to the reduction in crystal volume. This prevents a period of low temperature from not negatively and permanently affecting the structure of the powder and contributes to good handling properties of the composition. Efflorescent materials for use herein include sulphate and citrates, in one embodiment the efflorescent material is sodium sulphate.

Although enzyme granulates having low levels of efflorescent material are more prone to instability issues than enzyme granulates with high levels of efflorescent material even enzyme granulates with high levels of efflorescent material are prone to stability issues. This instability is especially true in stressed detergent compositions, such as phosphate free detergent compositions.

It has been surprisingly found that enzyme stability, and therefore detergent composition stability, is increased in phosphate free detergent compositions when enzyme granulates are combined with specific bleach particles that comprise a core and at least two coating layers. Specifically, the bleach particles comprise an inner layer of efflorescent materials at least partially enclosing the core, and an outer layer of water-insoluble materials at least partially enclosing the inner layer. In one embodiment, the bleach particle comprises a core substantially consisting of bleach, an inner layer comprising efflorescent materials, and an outer layer substantially comprising water-insoluble materials. In one embodiment, the outer coating layer substantially consists of silicate, in another embodiment, sodium silicate. By “substantially” it is meant that at least 90%, in one embodiment at least 95% and in another embodiment at least 99%, by weight of the referenced layer is the desired material, such as sodium silicate in the outer layer. Said sodium silicate has a silicate ratio of from about 2.5 to about 4.5, in another embodiment from about 2.9 to about 4, and in another embodiment from about 3.0 to about 3.4. Silicate ratio is defined as the ratio of Si0₂ to M₂0, and in the case of sodium silicate, M is Na (sodium).

It is believed that the stabilized bleach particles of this invention contribute to enzyme stability in a phosphate free detergent composition due to the structure of the bleach particle. In particular the protective layers are believed to work via two mechanisms:
(a) controlling moisture content, particularly reducing the free water content of the particle which in turn prevents hydrogen peroxide migration; and
(b) in the case of the insoluble coating reducing the exposed surface area through which water can migrate into the particle and hydrogen peroxide can migrate out of the particle.

### Coated Bleach Particle

The stabilized bleach particles of the detergent composition are coated bleach particles comprising a core and at least two coating layers. Specifically, the coated bleach particles comprise an inner layer of efflorescent materials at least partially enclosing the core and firmly adhering thereto, and an outer layer of water-insoluble materials at least partially enclosing the inner layer and firmly adhering thereto. In one embodiment, the bleach particle comprises a core substantially consisting of bleach, in one embodiment sodium percarbonate; an inner layer comprising efflorescent materials; and an outer layer substantially comprising water-insoluble materials, in one embodiment, sodium silicate.

The coated bleach particles comprise a core substantially consisting of bleach. In one embodiment, the core substantially consists of sodium percarbonate. The term “substantially” is taken to mean that, as a result of the production process, the core may contain small quantities of auxiliary substances, i.e. substances other than bleach. The auxiliary substances may be present in an amount of less than 10%, in another embodiment less than 5%, in another embodiment less than 1%, by weight of the core. The auxiliary substances may be active oxygen stabilisers, for example, silicates and/or magnesium compounds. The auxiliary substances may also be inorganic or organic compounds which are used as nuclei in fluidised bed spray granulation for the production of sodium per carbonate, for example, the production of soda.

In one embodiment, the coated bleach particles comprise an inner layer of efflorescent materials at least partially enclosing the core and firmly adhering thereto. The inner layer substantially consists of an efflorescent material which may be partially hydrated. Suitable efflorescent materials include sodium sulphate, sodium carbonate, and mixtures thereof. The bleach particle of the invention does not need a thick inner layer in order to provide stability benefits. In one embodiment, the inner layer is from about 3% to about 10%, in another embodiment from about 5% to about 8%, by weight of the total bleach particle.

In one embodiment, the coated bleach particles comprise an outer layer of water-insoluble materials at least partially enclosing the inner layer and firmly adhering thereto. The outer coating layer substantially consists of a water-insoluble material. Suitable water-insoluble materials include alkali metal silicate, in one embodiment, sodium silicate. Said sodium silicate has a silicate ratio of from about 2.5 to about 4.5, in another embodiment from about 2.9 to about 4, and in another embodiment from about 3 to about 3.4. By “water-insoluble” it is meant a material that has a solubility of less than 0.01 g/cm³ at a temperature of about 20° C. In one embodiment, the outer layer comprises from about 0.2% to about 1.5 wt. %, in another embodiment from about 0.5% to 1 wt. % sodium silicate.

It is believed that the outer layer of water-insoluble materials, in one embodiment silicate, offers sufficient encapsulation to provide stability benefits while also containing large enough defects in the outer layer that the bleach (in one embodiment, percarbonate), is released into the wash liquor in a desirable timeframe. In one embodiment, greater than 80% of the core substantially comprising bleach is released in less than 10 minutes, in another embodiment less than 7 minutes) into the wash liquor. Too thick of an outer layer delays release of the core (and therefore diminishes bleach performance) whereas too thin of an outer layer will not provide the stability benefits in the detergent composition.

In one embodiment, the water-insoluble outer layer is a thermally sensitive material that is solid at room temperature but melts in the temperature range of from about 30° C. to about 60° C., in another embodiment from about 35° C. to about 45° C. The outer layer can provide protection from water ingress during storage while being able to release the bleach core under typical automatic dishwashing wash conditions (40° C. to about 60° C. wash cycles).

In one embodiment, the coated particle may comprise one or more further coating layer(s), for example one or two further coating layers, in addition to the inner layer and the outer layer. The additional coating layer(s) substantially enclose the outer layer and adhere firmly thereto. The additional coating layer(s) may comprise water soluble salts, in one embodiment effloresecent agents such as sodium sulphate. It is believed that such additional coating layer(s) can provide protection for the water-insoluble outer layer from the impact and shear forces associated with mixing and conveying of powder products prior to packing. Such forces are capable of inducing cracking or chipping of the outer layer thereby rendering the bleach particles less effective at driving desired stability benefits. By providing additional coating layer(s) substantially enclosing the outer layer, the stability benefits of the bleaching particles can be better maintained.

Preparation of the coated bleach particles comprises coating processes which are known, in one embodiment, fluidized bed coating. Fluidized bed coating is characterized in that for the preparation of an outer shell layer comprising, for example alkali metal silicate, an aqueous solution containing alkali metal silicate with an alkali metal silicate concentration in the range from about 2% to about 20 wt. %, and a silicate ratio of greater than 2.5, is used. This solution is sprayed onto, for example, sodium percarbonate particles which have at least one inner layer comprising an efflorescent material. The spraying is carried out in a fluidized bed, with simultaneous evaporation of water, until the outer layer comprises from about 0.2% to about 1.5 wt. % alkali metal silicate.

So that good stabilising may be achieved, endeavours are taken during production to obtain a stabilized coated bleach particle having the lowest possible degree of hydration. For this reason, the fluidised bed temperature during application of the inner layer to the core and the outer layer to the inner layer is maintained above the transition temperature of the decahydrate (32.4° C.).

The resulting coated bleach particle has a weight geometric mean particle size of from about 400 μm to about 1200 μm, in one embodiment from about 500 μm to about 1000 μm, and in another embodiment from about 700 μm to about 900 μm. It is beneficial that the bleach particles have a low level of fine and coarse particles; in one embodiment less than 10% by weight of the bleach particles have a size above about 1400 μm, in another embodiment above 1200 μm or below about 400 μm, in another embodiment below about 200 μm. The mean particle size and particle size distribution further contributes to the stability of the detergent composition. In one embodiment, the coated bleach particle has a weight geometric mean particle size of from about 700 to about 1000 μm, with less than about 3% by weight of the bleach particle above about 1180 μm and less than about 5% by weight of the bleach particle below about 200 μm. The weight geometric mean particle size can be measured using a Malvern particle size analyser based on laser diffraction.

The detergent composition comprises from about 3% to about 30%, in another embodiment from about 5% to about 20%, and in another embodiment from about 7% to about 15%, bleach particle by weight of the composition.

### Stabilized Enzyme Particles

The stabilized enzyme particles of the detergent composition can have either a core/coating design wherein the enzyme particles comprise a central core and one or more coatings substantially surrounding the core, or a layered granule design made by a fluid bed process.

### A. Core/Coating Particles

Core/coating enzyme particles comprise a core substantially surrounded by one or more coatings. These one or more coatings reduce the risk of enzyme dust release as a result of abrasion, and further protect the enzyme core from ingress. In one embodiment, the core substantially comprises an enzyme. In another embodiment, the core may comprise salts, efflorescent agents, binding agents, kaolin/CaCO₃ and cellulose fibers, in addition to the enzyme. In one embodiment, the core comprises an enzyme and the efflorescent agent sodium sulphate. Enzymes suitable for use in the core are discussed in more detail below.

The one or more coatings on the enzyme particles may comprise polymers, pigments (to improve visual appearance), further excipients, antioxidants, and mixtures thereof. Suitable coatings include polymers such as polyethylene glycol, hydroxypropylmethylcellulose (HPMC), polyvinylalcohol (PVA), carboxymethyl cellulose, carboxymethyl cellulose, methyl cellulose, hydroxyethyl cellulose, hydroxypropyl cellulose and corresponding mixed ethers, gelatin, casein, polyacrylates, polymethacrylates, copolymers of acrylic acid with maleic acid, or vinyl group-containing compounds, partially saponified polyvinyl acetate and polyvinylpyrrolidone. In one embodiment, the polymer is a polyethylene glycol having a molecular weight of from about 300 to about 10,000, in another embodiment from about 2,000 to about 6,000. Suitable pigments may be agents that either provide a distinct colour or are whitening agents such as titanium dioxide. Suitable excipients include starches, sugars, sodium carbonate, calcium carbonate, silica, titania, alumina, clays such as bentonite, and/or talc. Suitable antioxidants may be selected from the group consisting of sodium sulphite, reducing sugars, ascorbic acid, tocopherol, gallates, thiosulfate, substituted phenols, hydroquinones, catechols, and aromatic amines and organic sulfides, polysulfides, dithiocarbamates, phosphites, phosphonates, vitamin E, catalase, low molecular weight peptides, and mixtures thereof. These antioxidants essentially act as sacrificial substrates to protect the enzyme particle.

In one embodiment, the coating comprises polyethylene glycol, kaolin, and titanium dioxide (white pigment). In one embodiment, a second coating of efflorescent agent, in one embodiment sodium sulphate, at least partially surrounds the coating comprising polyethylene glycol, kaolin, and titanium dioxide (white pigment). In one embodiment, the efflorescent agent is sodium sulphate and is present at a level of from about 30% to about 80%, or from about 40% to about 75%, or from about 50% to about 65%, by weight of the enzyme particle. Suitable core/coating designs include the grades sold as GT, Evity and GTX by Novozymes.

### B. Layered Granules

In another embodiment, the enzymes have a layered granule structure made via fluid bed processing. In one embodiment, the core comprises a central part substantially free of enzymes, and a layer surrounding the central part of the core comprising enzymes. The surrounding layer, in addition to comprising enzymes, may comprise other stabilizers such as antioxidants. In addition to the core comprising a central part and a surrounding layer, the enzyme particle may comprise a shell substantially contacting the surrounding layer. In one embodiment, the shell comprises a plurality of layers, the outer most layer of the granule being a protective layer. In one embodiment, the central part of the core and at least one of the layers of the shell comprises an efflorescent material.

The central part of the core comprises from about 1% to about 60%, in another embodiment from about 3% to about 50%, and another embodiment from about 5% to about 40% by weight of the total enzyme particle. In one embodiment, the central core is sodium sulphate. In one embodiment, the layer comprising the efflorescent material represents from about 0.5% to about 40%, in another embodiment from about 1% to about 30%, and in another embodiment from about 3% to about 20% by weight of the total enzyme particle. In one embodiment the most outer layer of the shell comprises polyvinyl alcohol, optionally titanium oxide (for aesthetic reasons) and combinations thereof. The protective layer of the shell comprises from about 0.05% to about 20%, in another embodiment from about 0.1% to about 15% and in another embodiment from about 1% to about 3% by weight of the total enzyme particle. The enzyme particle may also contain adjunct materials such as antioxidants, dyes, activators, solubilizers, binders, etc. Enzyme particles according to this embodiment can be made by a fluid bed layering process similar to that described in U.S. Pat. No. 5,324,649, U.S. Pat. No. 6,602,841 B1 and US2008/0206830A1.

Regardless of the process of making, the enzyme particles have a weight geometric mean particle size of from about 200 μm to about 1200 μm, in another embodiment from about 300 μm to about 1000 μm, and in another embodiment from about 400 μm to about 600 μm.

### C. Enzymes

Suitable enzymes for use in the enzyme particle include amylases, proteases, and mixtures thereof. In one embodiment, the enzyme is a protease, wherein the protease demonstrates at least 90%, in one embodiment at least 95%, in another embodiment at least 98%, in another embodiment at least 99%, and in a final embodiment 100% identity with the wild-type enzyme from Bacillus lentus. The protease comprises mutations in one or more, in another embodiment two or more, in another embodiment three or more, of the following positions using the BPN′ numbering system and amino acid abbreviations as illustrated in WO00/37627: 9, 15, 61, 68, 76, 87, 99, 101, 103, 104, 118, 128, 129, 130, 167, 170, 194, 205, 222 & 245 and optionally one or more insertions in the region comprising amino acids 95-103. The mutations are selected from one or more, in another embodiment two or more, and in another embodiment three or more of the following: V68A, N87S, S99D, S99SD, S99A, S101G, S103A, V104N/I, Y167A, R1705, A194P, V205I and/or M222S. The protease shows increased stability when combined with the silicate coated bleach particle of the invention as compared to being combined with an uncoated bleach particle.

In another embodiment, the enzyme particle comprises an amylase wherein the amylase is selected from the group comprising:
a) an amylase exhibiting at least 95% identity with the wild-type enzyme from Bacillus sp.707 (SEQ ID NO:7 in U.S. Pat. No. 6,093,562), especially those comprising one or more of the following mutations M202, M208, S255, R172, and/or M261, said amylase comprises one or more of M202L, M202V, M202S, M202T, M202I, M202Q, M202W, S255N and/or R172Q. In one embodiment, the amylase comprises the M202L or M202T mutations; and
b) an amylase exhibiting at least 95% identity with the wild-type enzyme from AA560 (SEQ ID NO. 12 in WO 06/002643), especially those comprising one or more of the following mutations 9, 26, 118, 149, 182, 186, 195, 202, 257, 295, 299, 320, 323, 339, 345 and 458 and optionally comprising one or more deletions at 183 and 184.

In one embodiment, the enzyme particle comprises a mixture of the protease described above and the amylase described above. This enzyme particle provides good cleaning and increased enzyme stability in the detergent composition.

Other proteases include metalloproteases and serine proteases, including neutral or alkaline microbial serine proteases, such as subtilisins (EC 3.4.21.62). Suitable proteases include those of animal, vegetable, or microbial origin. In one aspect, such suitable protease may be of microbial origin. The suitable proteases include chemically or genetically modified mutants of the aforementioned suitable proteases. In one aspect, the suitable protease may be a serine protease, such as an alkaline microbial protease or/and a trypsin-type protease. Examples of suitable neutral or alkaline proteases include:
(a) subtilisins (EC 3.4.21.62), including those derived from Bacillus, such as Bacillus lentus, B. alkalophilus, B. subtilis, B. amyloliquefaciens, Bacillus pumilus and Bacillus gibsonii described in U.S. Pat. No. 6,312,936 B1, U.S. Pat. No. 5,679,630, U.S. Pat. No. 4,760,025, U.S. Pat. No. 7,262,042 and WO09/021867.
(b) trypsin-type or chymotrypsin-type proteases, such as trypsin (e.g., of porcine or bovine origin), including the Fusarium protease described in WO 89/06270 and the chymotrypsin proteases derived from Cellumonas described in WO 05/052161 and WO 05/052146.
(c) metalloproteases, including those derived from Bacillus amyloliquefaciens described in WO 07/044993A2.

Proteases include those derived from Bacillus gibsonii or Bacillus Lentus.

In one embodiment, the protease is selected from the group comprising the below mutations (BPN′ numbering system) versus either the PB92 wild-type (SEQ ID NO:1) or the subtilisin 309 wild-type (SEQ ID NO:2).
(i) G118V+S128L+P129Q+S130A;
(ii) G118V+S128N+P129S+S130A+S166D;
(iii) G118V+S128L+P129Q+S130A+S166D;
(iv) G118V+S128V+P129E+S130K;
(v) G118V+S128V+P129M+S166D;
(vi) G118V+S128F+P129L+S130T
(vii) G118V+S128L+P129N+S130V
(viii) G118V+S128F+P129Q
(ix) G118V+S128V+P129E+S130K+S166D;
(x) S128R+P129Q+S130D
(xi) S128C+P129R+S130D
(xii) S128C+P129R+S130G
(xiii) S101G+V104N
(xiv) N76D+N87S+S103A+V104I;
(xv) V68A+N87S+S101G+V104N
(xvi) S99SD+S99A
(xvii) N87S+S99SD+S99A
(xviii) S9R+A15T+V68A+N218D+Q245R
(xix) S9R+A15T+V68A+H120N+N218D+Q245R
(xx) S9R+A15T+V68A+H120V+N218D+Q245R
(xxi) S9R+A15T+V68A+H120Q+N218D+Q245R
(xxii) S9R+A15T+V68A+N76D+Q245R
(xxiii) S9R+A15T+V68A+N218D+Q245R
(xxiv) S9R+A15T+V68A+N76D+N218D+Q245R
(xxv) S9R+A15T+V68A+Q245R
(xxvi) S9R+A15T+G61E+V68A+A98S+S99G+Q245R
(xxvii) S9R+A15T+G61E+V68A+A98S+S99G+N218D+Q245R

Suitable commercially available protease enzymes include those sold under the trade names Alcalase®, Savinase®, Primase®, Durazym®, Polarzyme®, Kannase®, Liquanase®, Ovozyme®, Neutrase®, Everlase®, Blaze® and Esperase® by Novozymes A/S (Denmark), those sold under the tradename Maxatase®, Maxacal®, Maxapem®, Properase®, Purafect®, Purafect Prime®, Purafect Ox®, FN3®, FN4®, Excellase® and Purafect OXP® by Genencor International, those sold under the tradename Opticlean® and Optimase® by Solvay Enzymes, those available from Henkel/Kemira, namely BLAP (sequence shown in FIG. 29 of U.S. Pat. No. 5,352,604 with the following mutations S99D+S101 R+S103A+V104I+G159S, hereinafter referred to as BLAP), BLAP R (BLAP with S3T+V4I+V199M+V205I+L217D), BLAP X (BLAP with S3T+V4I+V205I) and BLAP F49 (BLAP with S3T+V4I+A194P+V199M+V205I+L217D)—all from Henkel/Kemira; and KAP (Bacillus alkalophilus subtilisin with mutations A230V+S256G+S259N) from Kao. In one embodiment is a dual protease system, in particular a system comprising a protease comprising S99SD+S99A mutations (BPN′ numbering system) versus either the PB92 wild-type (SEQ ID NO:1) or the subtilisin 309 wild-type (SEQ ID NO:2) and a DSM14391 Bacillus Gibsonii enzyme, as described in WO 2009/021867 A2.

Levels of protease in the detergent composition include from about 0.1 mg to about 10 mg, from about 0.5 mg to about 5 mg, and from about 1 mg to about 4 mg of active protease per gram of the detergent composition.

In another embodiment, the enzyme for use herein includes alpha-amylases, including those of bacterial or fungal origin. Chemically or genetically modified mutants (variants) are included. In one embodiment, the amylase is an alkaline alpha-amylase derived from a strain of Bacillus, such as Bacillus licheniformis, Bacillus amyloliquefaciens, Bacillus stearothermophilus, Bacillus subtilis, or other Bacillus sp., such as Bacillus sp. NCIB 12289, NCIB 12512, NCIB 12513, DSM 9375 (U.S. Pat. No. 7,153,818) DSM 12368, DSMZ no. 12649, KSM AP1378 (WO 97/00324), KSM K36 or KSM K38 (EP 1,022,334). Amylases include:
(a) the variants described in WO 94/02597, WO 94/18314, WO96/23874 and WO 97/43424, especially the variants with substitutions in one or more of the following positions versus the enzyme listed as SEQ ID No. 2 in WO 96/23874: 15, 23, 105, 106, 124, 128, 133, 154, 156, 181, 188, 190, 197, 202, 208, 209, 243, 264, 304, 305, 391, 408, and 444.
(b) the variants described in U.S. Pat. No. 5,856,164 and WO99/23211, WO 96/23873, WO00/60060 and WO 06/002643, especially the variants with one or more substitutions in the following positions versus the AA560 enzyme (SEQ ID NO:3): 26, 30, 33, 82, 37, 106, 118, 128, 133, 149, 150, 160, 178, 182, 186, 193, 203, 214, 231, 256, 257, 258, 269, 270, 272, 283, 295, 296, 298, 299, 303, 304, 305, 311, 314, 315, 318, 319, 339, 345, 361, 378, 383, 419, 421, 437, 441, 444, 445, 446, 447, 450, 461, 471, 482, 484, preferably that also contain the deletions of D183* and G184*.
(c) variants exhibiting at least 90% identity with SEQ ID No. 4 in WO06/002643, the wild-type enzyme from Bacillus SP722, especially variants with deletions in the 183 and 184 positions and variants described in WO 00/60060, which is incorporated herein by reference.
(d) variants exhibiting at least 95% identity with the wild-type enzyme from Bacillus sp.707 (SEQ ID NO:4), especially those comprising one or more of the following mutations M202, M208, S255, R172, and/or M261. Preferably said amylase comprises one or more of M202L, M202V, M202S, M202T, M202I, M202Q, M202W, S255N and/or R172Q. In one embodiment, the amylase comprises the M202L or M202T mutations.

In one embodiment, -amylases include the below variants of SEQ ID NO: 3:
(a) one or more, in one embodiment two or more, in another embodiment three or more substitutions in the following positions: 9, 26, 149, 182, 186, 202, 257, 295, 299, 323, 339 and 345; and
(b) optionally with one or more, in another embodiment four or more of the substitutions and/or deletions in the following positions: 118, 183, 184, 195, 320 and 458, which if present comprise R118K, D183*, G184*, N195F, R320K and/or R458K.

Amylases include those comprising the following sets of mutations:
(i) M9L+,M323T;
(ii) M9L+M202L/T/V/I+M323T;
(iii) M9L+N195F+M202L/T/V/I+M323T;
(iv) M9L+R118K+D183*+G184*+R320K+M323T+R458K;
(v) M9L+R118K+D183*+G184*+M202L/T/V/I; R320K+M323T+R458K;
(vi) M9L+G149A+G182T+G186A+M202L+T257I+Y295F+N299Y+M323T+A339S+E345R;
(vii) M9L+G149A+G182T+G186A+M202I+T257I+Y295F+N299Y+M323T+A339S+E345R;
(viii) M9L+R118K+G149A+G182T+D183*+G184*+G186A+M202L+T257I+Y295F+N299Y+R320K+M323T+A339S+E345R+R458K;
(ix) M9L+R118K+G149A+G182T+D183*+G184*+G186A+M202I+T257I+Y295F+N299Y+R320K+M323T+A339S+E345R+R458K;
(x) M9L+R118K+D183*+D184*+N195F+M202L+R320K+M323T+R458K;
(xi) M9L+R118K+D183*+D184*+N195F+M202T+R320K+M323T+R458K;
(xii) M9L+R118K+D183*+D184*+N195F+M202I+R320K+M323T+R458K;
(xiii) M9L+R118K+D183*+D184*+N195F+M202V+R320K+M323T+R458K;
(xiv) M9L+R118K+N150H+D183*+D184*+N195F+M202L+V214T+R320K+M323T+R458K; or
(xv) M9L+R118K+D183*+D184*+N195F+M202L+V214T+R320K+M323T+E345N+R458K.
(xvi) M9L+R118K+G149A+G182T+D183*+G184*+G186A+N195F+M202L+T257I+Y295F+N299Y+R320K+M323T+A339S+E345R+R458K

Suitable commercially available alpha-amylases include DURAMYL®, LIQUEZYME®, TERMAMYL®, TERMAMYL ULTRA®, NATALASE®, SUPRAMYL®, STAINZYME®, STAINZYME PLUS®, POWERASE®, FUNGAMYL® and BAN® (Novozymes A/S, Bagsvaerd, Denmark), KEMZYM® AT 9000 Biozym Biotech Trading GmbH Wehlistrasse 27b A-1200 Wien Austria, RAPIDASE®, PURASTAR®, ENZYSIZE®, OPTISIZE HT PLUS® and PURASTAR OXAM® (Genencor International Inc., Palo Alto, Calif.) and KAM® (Kao, 14-10 Nihonbashi Kayabacho, 1-chome, Chuo-ku Tokyo 103-8210, Japan). Amylases especially preferred for use herein include NATALASE®, STAINZYME®, STAINZYME PLUS®, POWERASE® and mixtures thereof.

The composition of the invention comprises at least 0.01 mg of active amylase per gram of composition, in another embodiment from about 0.05 mg to about 10 mg, in another embodiment from about 0.1 mg to about 6 mg, in another embodiment from about 0.2 mg to about 4 mg of amylase per gram of composition.

In addition to the enzyme particle, the detergent composition as a whole may comprise other enzymes in addition to the protease and/or amylase selected from the group comprising hemicellulases, cellulases, cellobiose dehydrogenases, peroxidases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and mixtures thereof.

Cellulase enzymes are additional enzymes, in one embodiment, microbial-derived endoglucanases exhibiting endo-beta-1,4-glucanase activity (E.C. 3.2.1.4), including a bacterial polypeptide endogenous to a member of the genus Bacillus which has a sequence of at least 90%, in one embodiment at least 94%, in another embodiment at least 97% and in another embodiment at least 99% identity to the amino acid sequence SEQ ID NO:2 in U.S. Pat. No. 7,141,403B2 and mixtures thereof. Commercially available cellulases for use herein are Celluzyme®, Celluclean®, Whitezyme® (Novozymes A/S) and Puradax HA® and Puradax® (Genencor International).

### Amino Acid-Based Builders

Amino acid based builders include aminocarboxylic acids, salts, and derivatives thereof. In one embodiment, the aminocarboxylic builder is an aminopolycarboxylic builder, in another embodiment, a glycine-N,N-diacetic acid or derivative of general formula MOOC—CHR—N(CH₂COOM)₂ where R is C1-12 alkyl and M is alkali metal. In another embodiment, the aminocarboxylic builder for use herein is methylglycine diacetic acid (MGDA), in another embodiment alkali metal salts, in another embodiment sodium, potassium, and mixed sodium/potassium salts. In one embodiment is the tri-sodium salt, specifically the tri-sodium salt of MGDA.

The builder may be present as an encapsulate or a granulate such that its interactions with the bleach on storage are minimized.

In one embodiment, the aminocarboxylic builder is present in the composition in amorphous form. A builder is considered “amorphous” if at least 30%, in another embodiment at least 50%, at least 60% and at least 70% of the material, by weight thereof, is amorphous. In an amorphous material the atoms are arranged in a random way. In a crystalline material the atoms are arranged in a regular pattern. Amorphous materials lack a coherent, large-range structure. An amorphous material when subjected to XR diffraction at room temperature presents a very broad peak, as opposed to a crystalline material that presents a sharp narrow diffraction peak.

The builder may be a water-soluble salt selected from the group consisting of sulphate, citrate, carbonate, bicarbonate, silicate, and mixtures thereof. In one embodiment the salt is sodium sulphate. Burkeite is another water-soluble salt preferred for use herein.

It is believed that by using a detergent composition comprising coated bleach particles, oxidation of the amino acid-based builders during storage and the composition's associated yellowing can be minimized, particularly if the amino acid-based builders are either encapsulated in a separate compartment of a multi-compartment pouch, or if it is present in a granulate comprising protective efflorescent material and optionally one or more antioxidants, such as those described above.

### Cleaning Actives

Any cleaning active can be used as part of the product of the invention. The levels given are weight percent and refer to the total composition (excluding the enveloping water-soluble material, in the case of unit dose products having a wrapper or enveloping material). The composition is free of phosphate builder, and may comprise one or more detergent active components which may be selected from surfactants, bleach activators, bleach catalysts, alkalinity sources, dispersants, anti-corrosion agents and metal care agents.

### Surfactant

Surfactants suitable for use herein include non-ionic surfactants. The detergent composition of the invention is substantially free of anionic and cationic surfactants due to the fact that these types of surfactants cause too much sudsing during the automatic dishwashing process. Sudsing in automatic dishwashing processes are best avoided because they slow down, or even bring to a halt, the rotor of the dishwashing machine.

Traditionally, non-ionic surfactants have been used in automatic dishwashing detergents for surface modification purposes. In particular, non-ionic surfactants have been used for sheeting, to avoid filming and spotting, and to improve shine.

The composition of the invention comprises a non-ionic surfactant or a non-ionic surfactant system having a phase inversion temperature (as measured at a concentration of 1% in distilled water) of about 40° C. to about 70° C., in another embodiment of about 45° C. to about 65° C. A “non-ionic surfactant system” is meant herein as a mixture of two or more non-ionic surfactants. In one embodiment the detergent composition comprises a non-ionic surfactant systems for increased stability.

Phase inversion temperature is the temperature below which a surfactant, or a mixture thereof, partitions into the water phase. Phase inversion temperature can be determined visually by identifying at which temperature cloudiness occurs. The phase inversion temperature of a non-ionic surfactant or system can be determined as follows: a solution containing 1% of the corresponding surfactant or mixture by weight of the solution in distilled water is prepared. The solution is stirred gently before phase inversion temperature analysis to ensure that the process occurs in chemical equilibrium. The phase inversion temperature is taken in a thermostable bath by immersing the solutions in a 75 mm sealed glass test tube. To ensure the absence of leakage, the test tube is weighed before and after phase inversion temperature measurement. The temperature is gradually increased at a rate of less than 1° C. per minute, until the temperature reaches a few degrees below the pre-estimated phase inversion temperature. Phase inversion temperature is determined visually at the first sign of turbidity.

In one embodiment, the non-ionic surfactant is an alcohol alkoxylated surfactant. An alcohol alkoxylated surfactant is a compound obtained by the condensation of alkylene oxide groups with an organic hydrophobic material which may be aliphatic or alkyl aromatic in nature, in another embodiment is a compound selected from the group consisting of a C2-C18 alcohol alkoxylated surfactant having EO, PO and/or BO moieties. The moieties can be in block configuration or randomly distributed.

In one embodiment, the alcohol alkoxylated surfactant is substantially free of other alkoxylated groups (i.e. less than 10%, less than 5%, and less than 1% of alkoxylated groups other than ethoxy groups). Suitable herein are primary alcohols having from about 8 to 18 carbon atoms and on average from about 1 to 12 mol of ethylene oxide (EO) per mole of alcohol in which the alcohol radical may be linear or 2-methyl-branched, or may contain a mixture of linear and methyl-branched radicals, as are typically present in oxo alcohol radicals. In one embodiment, alcohol ethoxylates have linear radicals of alcohols of natural origin having from 12 to 18 carbon atoms, for example, of coconut, palm, tallow fat or oleyl alcohol, and on average from about 2 to 8 EO per mole of alcohol. Ethoxylated alcohols include, for example, C12-14-alcohols having 3 EO or 4 EO, C9-11-alcohols having 7 EO, C13-15-alcohols having 3 EO, 5 EO, 7 EO or 8 EO, C12-18-alcohols having 3 EO, 5 EO or 7 EO and mixtures thereof, such as mixtures of C12-14-alcohol having 3 EO and C12-18-alcohol having 5 EO. The degrees of ethoxylation specified are statistical average values which may be an integer or a fraction for a specific product. In one embodiment, the alcohol ethoxylates have a narrowed homolog distribution (narrow range ethoxylates, NRE). In addition to these surfactants, it is also possible to use fatty alcohols having more than 12 EO. Examples thereof are tallow fatty alcohols having 14 EO, 25 EO, 30 EO or 40 EO.

In one embodiment, non-ionic surfactants include the condensation products of alcohols having an alkyl group containing from about 8 to about 14 carbon atoms with an average of from about 6 to about 8 moles of ethylene oxide per mole of alcohol. At least about 25%, in another embodiment at least about 75% of the surfactant is a straight-chain ethoxylated primary alcohol. In one embodiment, the HLB (hydrophilic-lipophilic balance) of the alcohol alkoxylated surfactant is less than about 18, less than about 15, and less than about 14 HLB. Commercially available products for use herein include the Lutensol®TO series and the C13 oxo alcohol ethoxylated surfactants supplied by BASF.

Other suitable alcohol ethoxylated surfactants for use herein are C2-C18 alcohol alkoxylated surfactants having EO, PO and/or BO moieties having either random or block distribution. In one embodiment, the surfactant system comprises an ethoxylated alcohol having a C10-C16 alcohol having from 4 to 10 ethoxy groups. The alkoxylated alcohol is present at a level of from about 0.1% to about 20%, in another embodiment from about 1% to about 10%, and in another embodiment from about 4% to about 8% by weight of the detergent composition.

Other suitable alkoxylated alcohols for use herein include a C2-C18 alcohol alkoxylate having EO, PO and/or BO moieties, specially a C2-C18 alcohol comprising EO and BO moieties in a random configuration. Fatty alcohol alkoxylates are Adekanol B2020 (Adeka), Dehypon LS36 (Cognis), Plurafac LF 221 (C13-15, EO/BO (95%)), Plurafac LF 300, Plurafac LF 303 (EO/PO), Plurafac LF 1300, Plurafac LF224, Degressal SD 20 (polypropoxylate) (all from BASF), Surfonic LF 17 (C12-18 ethoxylated propoxylated alcohol, Huntsman), Triton EF 24 (Dow), Neodol ethoxylates from Shell.

Also suitable for use herein are polyoxyalkene condensates of aliphatic carboxylic acids, whether linear- or branched-chain and unsaturated or saturated, especially ethoxylated and/or propoxylated aliphatic acids containing from about 8 to about 18 carbon atoms in the aliphatic chain and incorporating from about 2 to about 50 ethylene oxide and/or propylene oxide units. Suitable carboxylic acids include “coconut” fatty acids (derived from coconut oil) which contain an average of about 12 carbon atoms, “tallow” fatty acids (derived from tallow-class fats) which contain an average of about 18 carbon atoms, palmitic acid, myristic acid, stearic acid and lauric acid.

Also suitable for use herein are polyoxyalkene condensates of aliphatic alcohols, whether linear- or branched-chain and unsaturated or saturated, especially ethoxylated and/or propoxylated aliphatic alcohols containing from about 6 to about 24 carbon atoms and incorporating from about 2 to about 50 ethylene oxide and/or propylene oxide units. Suitable alcohols include “coconut” fatty alcohol, “tallow” fatty alcohol, lauryl alcohol, myristyl alcohol and oleyl alcohol.

Other example types of nonionic surfactants are linear fatty alcohol alkoxylates with a capped terminal group, as described in U.S. Pat. No. 4,340,766 to BASF.

Other types include olyoxyethylene -polyoxypropylene block copolymers having formula:

HO(CH2CH2O)a(CH(CH3)CH2O)b(CH2CH2O)cH; or

HO(CH(CH3)CH2O)d(CH2CH2O)e(CH(CH3)CH2O)H

wherein a, b, c, d, e and f are integers from 1 to 350 reflecting the respective polyethylene oxide and polypropylene oxide blocks of said polymer. The polyoxyethylene component of the block polymer constitutes at least about 10% of the block polymer. The material can for instance have a molecular weight of between about 1,000 and about 15,000, more specifically from about 1,500 to about 6,000. These materials are well-known in the art. They are available under the trademark “Pluronic” and “Pluronic R”, from BASF Corporation.

Suitable nonionic surfactants include: i) ethoxylated non-ionic surfactants prepared by the reaction of a monohydroxy alkanol or alkyphenol with 6 to 20 carbon atoms with at least 12 moles, in another embodiment at least 16 moles, and in another embodiment at least 20 moles of ethylene oxide per mole of alcohol or alkylphenol; ii) alcohol alkoxylated surfactants having a from 6 to 20 carbon atoms and at least one ethoxy and propoxy group. In one embodiment is a mixture of surfactants (i) and (ii).

Other suitable non-ionic surfactants are epoxy-capped poly(oxyalkylated) alcohols represented by the formula:

R1O[CH2CH(CH3)O]x[CH2CH2O]y[CH2CH(OH)R2] (I)

wherein R1 is a linear or branched aliphatic hydrocarbon radical having from about 4 to about 18 carbon atoms; R2 is a linear or branched aliphatic hydrocarbon radical having from about 2 to about 26 carbon atoms; x is an integer having an average value of from about 0.5 to about 1.5, in another embodiment about 1; and y is an integer having a value of at least 15, in another embodiment at least 20.

In the surfactant of formula I, at least about 10 carbon atoms are in the terminal epoxide unit [CH2CH(OH)R2]. Suitable surfactants of formula I, according to the present invention, are Olin Corporation's POLY-TERGENT® SLF-18B nonionic surfactants, as described, for example, in WO 94/22800, published Oct. 13, 1994 by Olin Corporation.

Non-ionic surfactants and/or systems have a Draves wetting time of less than 360 seconds, in one embodiment less than 200 seconds, in another embodiment less than 100 seconds, and in another embodiment less than 60 seconds as measured by the Draves wetting method (standard method ISO 8022 using the following conditions; 3-g hook, 5-g cotton skein, 0.1% by weight aqueous solution at a temperature of 25° C.).

Amine oxides surfactants also useful in the present invention as anti-redeposition surfactants include linear and branched compounds having the formula:
O—
   |R3(OR4)xN+(R5)2
   wherein R3 is selected from an alkyl, hydroxyalkyl, acylamidopropoyl and alkyl phenyl group, or mixtures thereof, containing from about 8 to about 26 carbon atoms, in another embodiment from about 8 to about 18 carbon atoms; R4 is an alkylene or hydroxyalkylene group containing from about 2 to about 3 carbon atoms, in another embodiment from about 2 carbon atoms, or mixtures thereof; x is from 0 to 5, in another embodiment from 0 to 3; and each R5 is an alkyl or hydroxyalkyl group containing from 1 to 3, in another embodiment from 1 to 2 carbon atoms, or a polyethylene oxide group containing from 1 to 3, in one embodiment 1, ethylene oxide groups. The R5 groups can be attached to each other, e.g., through an oxygen or nitrogen atom, to form a ring structure.

These amine oxide surfactants in particular include C10-C18 alkyl dimethyl amine oxides and C8-C18 alkoxy ethyl dihydroxyethyl amine oxides. Examples of such materials include dimethyloctylamine oxide, diethyldecylamine oxide, bis-(2-hydroxyethyl)dodecylamine oxide, dimethyldodecylamine oxide, dipropyltetradecylamine oxide, methylethylhexadecylamine oxide, dodecylamidopropyl dimethylamine oxide, cetyl dimethylamine oxide, stearyl dimethylamine oxide, tallow dimethylamine oxide and dimethyl-2-hydroxyoctadecylamine oxide. In one embodiment are C10-C18 alkyl dimethylamine oxide, and C10-18 acylamido alkyl dimethylamine oxide.

Non-ionic surfactants may be present in amounts from about 0% to about 10%, in another embodiment from about 0.1% to about 10%, and in another embodiment from about 0.25% to about 6% by weight of the total composition.

The detergent composition is substantially free of anionic and cationic surfactants. By “substantially free of” it means that there is no deliberately added anionic and cationic surfactants. Such detergent compositions are desirable as both anionic and cationic surfactants can negatively impact cleaning and shine profile, particularly those surfactants that generate significant foam.

### Organic Polymers

The polymer, if present, is used in any suitable amount of from about 0.1% to about 50%, in another embodiment from about 0.5% to about 20%, in another embodiment from about 1% to about 10% by weight of the composition.

Organic polymers herein include acrylic acid containing polymers such as Sokalan PA30, PA20, PA15, PA10 and Sokalan CP10 (BASF GmbH), Acusol 45N, 480N, 460N (Rohm and Haas), acrylic acid/maleic acid copolymers such as Sokalan CP5 and acrylic/methacrylic copolymers. Soil release polymers herein include alkyl and hydroxyalkyl celluloses (U.S. Pat. No. 4,000,093), polyoxyethylenes, polyoxypropylenes and copolymers thereof, and nonionic and anionic polymers based on terephthalate esters of ethylene glycol, propylene glycol and mixtures thereof.

In one embodiment, sulfonated/carboxylated polymers are present for use in the composition of the invention. Suitable sulfonated/carboxylated polymers described herein may have a weight average molecular weight of less than or equal to about 100,000 Da, or less than or equal to about 75,000 Da, or less than or equal to about 50,000 Da, or from about 3,000 Da to about 50,000, in one embodiment from about 5,000 Da to about 45,000 Da.

As noted herein, the sulfonated/carboxylated polymers may comprise (a) at least one structural unit derived from at least one carboxylic acid monomer having the general formula (I):

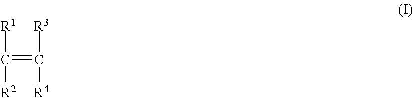

wherein R1 to R4 are independently hydrogen, methyl, carboxylic acid group or CH2COOH and wherein the carboxylic acid groups can be neutralized; (b) optionally, one or more structural units derived from at least one nonionic monomer having the general formula (II):

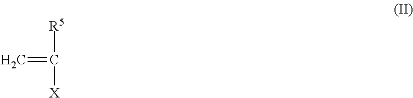

wherein R5 is hydrogen, C1 to C6 alkyl, or C1 to C6 hydroxyalkyl, and X is either aromatic (with R5 being hydrogen or methyl when X is aromatic) or X is of the general formula (III):

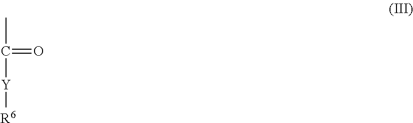

wherein R6 is (independently of R5) hydrogen, C1 to C6 alkyl, or C1 to C6 hydroxyalkyl, and Y is O or N; and at least one structural unit derived from at least one sulfonic acid monomer having the general formula (IV):

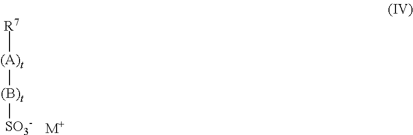

wherein R7 is a group comprising at least one sp2 bond, A is O, N, P, S or an amido or ester linkage, B is a mono- or polycyclic aromatic group or an aliphatic group, each t is independently 0 or 1, and M+ is a cation. In one aspect, R7 is a C2 to C6 alkene. In another aspect, R7 is ethene, butene or propene.

Carboxylic acid monomers include one or more of the following: acrylic acid, maleic acid, itaconic acid, methacrylic acid, or ethoxylate esters of acrylic acids, acrylic and methacrylic acids. Sulfonated monomers include one or more of the following: sodium (meth) allyl sulfonate, vinyl sulfonate, sodium phenyl (meth) allyl ether sulfonate, or 2-acrylamido-methyl propane sulfonic acid. Non-ionic monomers include one or more of the following: methyl (meth) acrylate, ethyl (meth) acrylate, t-butyl (meth) acrylate, methyl (meth) acrylamide, ethyl (meth) acrylamide, t-butyl (meth) acrylamide, styrene, or α-methyl styrene.

The polymer comprises the following levels of monomers: from about 40% to about 90%, in one embodiment from about 60% to about 90%, by weight of the polymer of one or more carboxylic acid monomer; from about 5% to about 50%, in one embodiment from about 10% to about 40%, by weight of the polymer of one or more sulfonic acid monomers; and optionally from about 1% to about 30%, in one embodiment from about 2% to about 20% by weight of the polymer of one or more non-ionic monomers. In one embodiment the polymer comprises about 70% to about 80% by weight of the polymer of at least one carboxylic acid monomer and from about 20% to about 30% by weight of the polymer of at least one sulfonic acid monomer.

In one embodiment the carboxylic acid is (meth)acrylic acid. The sulfonic acid monomer is one of the following: 2-acrylamido methyl-1-propanesulfonic acid, 2-methacrylamido-2-methyl-1-propanesulfonic acid, 3-methacrylamido-2-hydroxypropanesulfonic acid, allysulfonic acid, methallysulfonic acid, allyloxybenzenesulfonic acid, methallyloxybenzensulfonic acid, 2-hydroxy-3-(2-propenyloxy)propanesulfonic acid, 2-methyl-2-propene-1-sulfonic acid, styrene sulfonic acid, vinylsulfonic acid, 3-sulfopropyl acrylate, 3-sulfopropyl methacrylate, sulfomethylacrylamid, sulfomethylmethacrylamide, and water soluble salts thereof. The unsaturated sulfonic acid monomer is 2-acrylamido-2-propanesulfonic acid (AMPS).

Commercial available polymers include: Alcosperse 240, Aquatreat AR 540 and Aquatreat MPS supplied by Alco Chemical; Acumer 3100, Acumer 2000, Acusol 587G and Acusol 588G supplied by Rohm & Haas; Goodrich K-798, K-775 and K-797 supplied by BF Goodrich; and ACP 1042 supplied by ISP technologies Inc. In one embodiment the polymers are Acusol 587G and Acusol 588G supplied by Rohm & Haas.

In the polymers, all or some of the carboxylic or sulfonic acid groups can be present in neutralized form, i.e. the acidic hydrogen atom of the carboxylic and/or sulfonic acid group in some or all acid groups can be replaced with metal ions, alkali metal ions and sodium ions.

Other suitable organic polymers for use herein include a polymer comprising an acrylic acid backbone and alkoxylated side chains, said polymer having a molecular weight of from about 2,000 to about 20,000, and said polymer having from about 20 wt % to about 50 wt % of an alkylene oxide. The polymer should have a molecular weight of from about 2,000 to about 20,000, or from about 3,000 to about 15,000, or from about 5,000 to about 13,000. The alkylene oxide (AO) component of the polymer is generally propylene oxide (PO) or ethylene oxide (EO) and generally comprises from about 20 wt % to about 50 wt %, or from about 30 wt % to about 45 wt %, or from about 30 wt % to about 40 wt % of the polymer. The alkoxylated side chains of the water soluble polymers may comprise from about 10 to about 55 AO units, or from about 20 to about 50 AO units, or from about 25 to 50 AO units. The polymers, in one embodiment water soluble, may be configured as random, block, graft, or other known configurations. Methods for forming alkoxylated acrylic acid polymers are disclosed in U.S. Pat. No. 3,880,765.

Other suitable organic polymers for use herein include polyaspartic acid (PAS) derivatives as described in WO 2009/095645 A1.

### Silicates

Silicates for use in the composition are sodium silicates such as sodium disilicate, sodium metasilicate and crystalline phyllosilicates. Silicates, if present, are at a level of from about 1% to about 20%, or from about 5% to about 15%, by weight of composition.

### Additional Bleach

In addition to the bleach particle essential for the composition of the invention, the composition can also comprise other types of bleach, such as organic bleach.

Typical organic bleaches are organic peroxyacids including diacyl and tetraacylperoxides, especially diperoxydodecanedioc acid, diperoxytetradecanedioc acid, and diperoxyhexadecanedioc acid. Dibenzoyl peroxide is a organic peroxyacid herein. Mono- and diperazelaic acid, mono- and diperbrassylic acid, and Nphthaloylaminoperoxicaproic acid are also suitable herein.

The diacyl peroxide, especially dibenzoyl peroxide, should be present in the form of particles having a weight average diameter of from about 0.1 to about 100 microns, or from about 0.5 to about 30 microns, or from about 1 to about 10 microns. At least about 25%, in another embodiment at least about 50%, in another embodiment at least about 75%, in another embodiment at least about 90%, of the particles are smaller than 10 microns, or smaller than 6 microns. Diacyl peroxides within the above particle size range have also been found to provide better stain removal especially from plastic dishware, while minimizing undesirable deposition and filming during use in automatic dishwashing machines. The diacyl peroxide particle size thus allows the formulator to obtain good stain removal with a low level of diacyl peroxide, which reduces deposition and filming. Conversely, as diacyl peroxide particle size increases, more diacyl peroxide is needed for good stain removal, which increases deposition on surfaces encountered during the dishwashing process.

Further typical organic bleaches include the peroxy acids, examples being the alkylperoxy acids and the arylperoxy acids. Representatives are (a) peroxybenzoic acid and its ring-substituted derivatives, such as alkylperoxybenzoic acids, but also peroxy-α-naphthoic acid and magnesium monoperphthalate, (b) the aliphatic or substituted aliphatic peroxy acids, such as peroxylauric acid, peroxystearic acid, ε-phthalimidoperoxycaproic acid[phthaloiminoperoxyhexanoic acid (PAP)], o-carboxybenzamidoperoxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-diperoxyazelaic acid, diperoxysebacic acid, diperoxybrassylic acid, the diperoxyphthalic acids, 2-decyldiperoxybutane-1,4-dioic acid, N,N-terephthaloyldi(6-aminopercaproic acid).

### Bleach Activators

Bleach activators are usually organic peracid precursors that enhance the bleaching action of dishware at dishwashing machine temperatures of 60° C. and below. Bleach activators suitable for use herein include compounds which, under perhydrolysis conditions, give rise to aliphatic peroxoycarboxylic acids having from 1 to 10 carbon atoms, or from 2 to 4 carbon atoms, and/or optionally substituted perbenzoic acid. Suitable substances bear O-acyl and/or N-acyl groups of the number of carbon atoms specified and/or optionally substituted benzoyl groups. In one embodiment the bleach activator is a polyacylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic anhydrides, in particular phthalic anhydride, acylated polyhydric alcohols, in particular triacetin, ethylene glycol diacetate and 2,5-diacetoxy-2,5-dihydrofuran and also triethylacetyl citrate (TEAC).

Bleach activators, if included in the composition, are present at a level of from about 0.1% to about 10%, or from about 0.5% to about 2%, by weight of the total composition.

### Bleach Catalyst

Bleach catalysts for use herein include a manganese complex, e.g. Mn-Me TACN, as described in EP 458 397 A; Co, Cu, Mn and Fe bispyridylamine and related complexes (U.S. Pat. No. 5,114,611); and pentamine acetate cobalt(III) and related complexes (U.S. Pat. No. 4,810,410). A complete description of bleach catalysts suitable for use herein can be found in WO 99/06521, pages 34, line 26 to page 40, line 16. The preferred bleach catalyst for use herein is a manganese complex, e.g. Mn-Me TACN, as described in EP 458 397 A.

Bleach catalysts, if included in the composition, are present at a level of from about 0.000%1 to about 2%, or from about 0.001% to about 1%, by weight of the total composition.

### Metal Care Agents

Metal care agents may be included in the composition to prevent or reduce the tarnishing, corrosion, or oxidation of metals, including aluminium, stainless steel and non-ferrous metals, such as silver and copper. Suitable examples include one or more of the following:
(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain C1-C20-alkyl groups and hydroxyl, thio, phenyl or halogen such as fluorine, chlorine, bromine and iodine.
(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K2TiF6, K2ZrF6, CoSO4, Co(NO3)2 and Ce(NO3)3, zinc salts, for example zinc sulphate, hydrozincite or zinc acetate.
(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. In one embodiment, the metal care agent is a zinc salt.

If present, the composition of the invention comprises from about 0.1% to about 5%, or from about 0.2% to about 4%, or from about 0.3% to about 3% by weight of the total composition of a metal care agent.

### Water-Soluble Pouch

In one embodiment, the product of the invention is a unit-dose product. Products in unit dose form include tablets, capsules, sachets, pouches, etc. In one embodiment, the unit dose is contained in a water-soluble film (including tablets, capsules, sachets, pouches). In one embodiment, the product is in the form of a water soluble pouch.

In one embodiment, the composition of the invention is contained in a water-soluble film pouch or a water soluble injection molded pouch. Examples of injection molded pouches can be found in U.S. 2011/0175257. The weight of the composition of the invention contained in the pouch is from about 10 to about 35 grams, in one embodiment from about 12 to about 26 grams, and in another embodiment from 14 to 22 grams. In the cases of unit dose pouches having a water-soluble material containing the detergent composition, the water-soluble material is not considered part of the composition.

In one embodiment, the pouches comprise one compartment. In another embodiment, the pouches comprise at least two side-by-side compartments to form multi-compartment pouches. In one embodiment, the two compartments are superposed to one another. The compartments contain components of a single claimed composition herein. Examples of multi-compartment pouches and the methods of making them can be found in U.S. Pat. No. 7,125,828.

In one embodiment, at least one of the compartments contains a powder component and the other compartment contains a non-powder component. Non-powder components can be in the form of a gel or a liquid. The powder component can be compressed powder or non-compressed powder or mixtures thereof. In one embodiment, at least one of the compartments contains a solid composition and another compartment contains a non-solid composition. In another embodiment, at least one of the compartments contains a solid composition and another compartment contains an aqueous liquid composition. The compartments can have the same or varying weight ratios.

In one embodiment, the two side-by-side compartments contain liquid compositions. In another embodiment, the compartments contain different compositions, and at least one compartment contains a solid composition. In one embodiment the solid composition is in powder form, specifically a densified powder. The solid composition contributes to the strength and robustness of the pack. In one embodiment, at least one compartment contains a multiphase composition.

In one embodiment, the pouch has an overall volume of from about 5 to about 70 ml, in another embodiment from about 15 to about 60 ml, in another embodiment from about 18 to 57 ml, and a longitudinal/transverse aspect ratio in the range from about 2:1 to about 1:8, in another embodiment from about 1:1 to about 1:4. The longitudinal dimension is defined as the maximum height of the pouch when the pouch is lying on one of the bases which has the maximum footprint with the pouch compartments superposed in a longitudinal direction, i.e. one over another, and under a static load of about 2 Kg. The transverse dimension is defined as the maximum width of the pouch in a plane perpendicular to the longitudinal direction under the same conditions. These dimensions are adequate to fit the dispensers of the majority of dishwashers. Although the shape of the pouch can vary widely, in order to maximize the available volume, pouches should have a base as similar as possible to the footprint of the majority of the dispensers, that is generally rectangular.

The enzymes can lose stability in the composition due to their interactions with bleach and builders (they can destabilize the enzyme by binding to the calcium of the enzymes). In addition, the performance of enzymes in a composition can be impaired by the alkalinity of the solution, bleach, builders, etc. In one embodiment, the solid composition comprises bleach and the liquid composition comprises enzymes. In one embodiment one of the films enclosing the enzyme-comprising composition dissolves prior to the films enclosing the bleach-containing composition during the main-wash cycle of the automatic dishwashing machine, thereby releasing the enzyme-containing composition into the wash liquor prior to the delivery of the bleach-containing composition. This gives the enzymes the possibility to operate under optimum conditions, avoiding interactions with other detergent actives.

Controlled release of the ingredients of the multi-compartment pouch can be achieved by modifying the thickness of the film and/or the solubility of the film material. The solubility of the film material can be delayed by, for example, cross-linking the film as described in WO 02/102,955 at pages 17 and 18. Other water-soluble films designed for rinse release are described in U.S. Pat. No. 4,765,916 and U.S. Pat. No. 4,972,017.

Other means of obtaining delayed release by multi-compartment pouches with different compartments, where the compartments are made of films having different solubility are taught in WO 02/08380.

### EXAMPLES

Abbreviations Used in the Examples

In the examples, the abbreviated component identifications have the following meanings:

Silicate: Amorphous Sodium Silicate (SiO2:Na2O=from 2:1 to 4:1)

Carbonate: Anhydrous sodium carbonate

Citrate: Sodium citrate dihydrate

Percarbonate: Sodium percarbonate

TAED: Tetraacetylethylenediamine

LF224: Non-ionic surfactant available from BASF

DPG: Dipropylene glycol
Neodol 1-9: Non-ionic surfactant available from available from Shell Chemical Company

I. Preparation of Test Compositions

The following test composition was prepared

| Ingredient | Level (% wt) |
|---|---|
| Solid composition | |
| Sodium Carbonate, granular | 43.21 |
| Sodium Sulphate | 10.91 |
| Stainzyme Plus ® (14.4 mg/g active) | 0.80 |
| Ultimase ® (100 mg/g active) | 2.06 |
| Bleach Activator (2% active) | 0.58 |
| TAED (92% active) | 3.95 |
| Polymer | 7.82 |
| Plurafac LF224 | 0.62 |
| Benzotriaole | 0.05 |
| HEDP (84% active) | 0.72 |
| MGDA (78% active) | 17.09 |
| Liquid composition | |
| Lutensol TO7 | 14.00 |
| Dipropylene glycol | 20.00 |
| Water | 5.50 |
| SLF 180 | 57.00 |
| Dye | 3.50 |
| Processing Aids | Balance |

The detergent composition above contains no sodium percarbonate—the sodium percarbonate is added as per the levels and types shown below.

The following formulations were tested:

Example 1 contained Ultimase® protease granule, containing >30% sulphate coating and sodium percarbonate particle A, with a stabilized bleach coating containing 0.75% sodium silicate and 6% sodium sulphate

Example 2 contained Ultimase® protease granule, containing >30% sulphate coating and sodium percarbonate particle B, with a coating of 6% sodium sulphate;

Each percarbonate from Examples 1 and 2 was added to the base detergent at a level of 1.868 g active (based on an Available Oxygen level of 13.4%), as detailed in the test compositions below.

| Example | Composition |
|---|---|
| Comparative Example 1 | Formulation as above + Percarbonate (A) |
| Comparative Example 2 | Formulation as above + Percarbonate (B) |

II. Sample Preparation

Samples were pre-weighed and pouched using 14.63 g of solid composition with the relevant amount of percarbonate to a total weight of 16.5 g. Within the pouch, 2.2 g of liquid composition was added to a single liquid top pouch above the powder compartment. A polvinyl alcohol film was used to pouch both the powder composition and the liquid composition to form one pouch.

III. Test Procedure

Testing Conditions
Storage temperature and humidity: 32° C./80° RH
Sample analysis timing: Initial samples before storage (reference), 4 week samples and 8 week samples.
Sample storage: 4 internal replicates per test leg placed in a 15 pouch bag with 7 Cascade Complete® unit dose pouches.

Four pouches of each test leg were labelled with unique identities as above (A & B), placed in a clear mixed Polyethylene/Polyethylene terephthalate plastic sealable bag, having dimensions width 180 mm×height 260 mm×gusset depth 45 mm, with seven additional Cascade Complete® pouches, a product of Procter and Gamble, USA. A total of 15 pouches (4 from each of the 2 test legs and the 7 control pouches) were added to the one bag ensuring that they experienced the same test conditions.

This was repeated once more. Each bag was sealed using a heat sealer and placed into a controlled 32° c/80° RH oven. Samples were removed after 4 and 8 weeks. After removal from the storage oven, the four test pouch samples per leg, were removed from the bag and the powder composition removed from each pouch. Two replicates were analysed for active Protease enzyme content and two replicates were analysed for bleach, available oxygen content. Two pouches of each test leg were analysed freshly made (initial sample before storage) for active Protease enzyme content and two further replicates for bleach, available oxygen analysis. Each test sample, post storage, was analysed and then compared to its un-stored respective initial sample (reference).

IV. Analysis of Protease Enzyme in Each Sample

In order to determine the protease activity, the hydrolysis of N-succinyl-L-alanyl-L-alanyl-L-prolyl-L-phenyl-p-nitroanilide (suc-AAPF-pNA) was measured. The reagent solutions used were: 100 mM Tris/HCl, pH 8.6, containing 0.005% TWEEN®-80 (Tris dilution buffer); 100 mM Tris buffer, pH 8.6, containing 1 mM CaCl2 and 0.005% TWEEN®-80 (Tris/Ca buffer); and 160 mM suc-AAPF-pNA in DMSO (suc-AAPF-pNA stock solution) (Sigma: S-7388). To prepare a suc-AAPF-pNA working solution, 1 ml suc-AAPF-pNA stock solution was added to 100 ml Tris/Ca buffer and mixed well for at least 10 seconds. The assay was performed by adding 10 l of diluted protease solution to each well of a 96-well MTP, immediately followed by the addition of 190 μl of 1 mg/ml suc-AAPF-pNA working solution. The solutions were mixed for 5 sec, and the absorbance change in kinetic mode (25 readings in 5 minutes) was read at 405 nm in an MTP reader, at 25° C.

V. Analysis of Bleach in Each Sample

Bleach analysis can be achieved by standard methods well known in the art, for example titrimetric methods that involve oxidation if iodide and back titration with sodium thiosulphate to quantify the iodine produced.

VI. Enzyme Stability Results After 4 & 8 Weeks Storage

| Protease enzyme analysis: % remaining post storage | Residual activity | |
|---|---|---|
| error = +/− 10% of value quoted | 4 week | 8 week |
| Comparative Example 1 | 100 | 91 |
| Comparative Example 2 | 66 | 36 |

The stability of comparative Example 1 containing protease and percarbonate A with a stabilized bleach coating containing 0.75% sodium silicate and 6% sodium sulphate is significantly superior after 4 & 8 weeks storage, versus comparative Example 2 containing protease and percarbonate B with 6% sodium sulphate coating.

VI. Bleach Stability Results After 8 Weeks Storage

| Bleach analysis: % remaining post storage | Residual activity |
|---|---|
| error = +/− 3% of value quoted | 8 week |
| Comparative Example 1 | 93 |
| Comparative Example 2 | 82 |

The stability of comparative Example 1 containing protease and percarbonate A with a stabilized bleach coating containing 0.75% sodium silicate and 6% sodium sulphate is significantly superior after 8 weeks storage, versus comparative Example 2 containing protease and percarbonate B with 6% sodium sulphate coating.

VII. Product Appearance; Development of Visible Powder Yellowing After Storage

| Product Appearance | |
|---|---|
| after 4 & 8 weeks Storage | Visible result |
| Visual assessment - | |
| versus the unstored references | |
| Reference samples | No visible signs of powder yellowing |
| (unstored initial, examples 1 & 2) | |
| Comparative Example 1 | No visible signs of powder yellowing |
| Comparative Example 2 | Visible appearance of powder yellowing. |

The product yellowing appearance of comparative Example 1 containing protease and percarbonate A with a stabilized bleach coating containing 0.75% sodium silicate and 6% sodium sulphate is significantly superior after 4 & 8 weeks storage, versus comparative Example 2 containing protease and percarbonate B with 6% sodium sulphate coating.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A phosphate-free automatic dishwashing detergent composition comprising:
a) a coated bleach particle comprising at least two layers, where in the coated bleach particle comprises:
(i) a core substantially consisting of bleach; and
(ii) an inner layer at least partially enclosing the core, wherein the inner layer comprises an efflorescent material; and
(iii) an outer layer at least partially enclosing the inner layer, wherein the outer layer comprises a water-insoluble material; and one or more of:
b) an enzyme particle comprising an efflorescent material and an enzyme, wherein the enzyme is selected from the group consisting of amylase, protease, and mixtures thereof; and
c) an amino acid-based builder selected from the group consisting of methyl-glycine-diacetic acid and salts, glutamic-N,N-diacetic acid and salts, and mixtures thereof; and
wherein the composition is substantially free of anionic and cationic surfactants.

2. A detergent composition according to claim 1, wherein the water-insoluble material is sodium silicate.

3. A detergent composition according to claim 1, wherein the bleach particle core is produced by fluidised bed spray granulation and the coating layer is obtainable by spraying an aqueous sodium silicate solution onto the core of the bleach particle in the fluidised bed and by evaporating water while maintaining a fluidised bed temperature of from about 35° C. to about 100° C.

4. A detergent composition according to claim 1, wherein the inner layer of the bleach particle is from about 5% to about 12% by weight of the particle.

5. A detergent composition according to claim 1, wherein the enzyme is a protease and wherein the protease demonstrates at least 90% identity with the enzyme of SEQ ID NO:2, comprising mutations in one or more of the following positions: 9, 15, 61, 68, 76, 87, 99, 101, 103, 104, 118, 128, 129, 130, 167, 170, 194, 205, 222 & 245 and optionally one or more insertions in the region comprising amino acids 95 to 103.

6. A detergent composition according to claim 1, wherein the enzyme is an amylase and wherein the amylase is selected from the group consisting of:
a) an amylase exhibiting at least 95% identity with SEQ ID NO:4 including those comprising one or more of the following mutations M202, M208, S255, R172, and/or M261; and
b) an amylase exhibiting at least 95% identity with SEQ ID NO. 3 including those comprising one or more of the following mutations 9, 26, 118, 149, 182, 186, 195, 202, 257, 295, 299, 320, 323, 339, 345 and 458 and further comprising one or more deletions at 183 and 184.

7. A detergent composition according to claim 1, further comprising a bleach activator.

8. A detergent composition according to claim 1, further comprising a dispersant selected from the group consisting of organic polymers, organic builders and mixtures thereof.

9. A detergent composition according to claim 8, wherein the composition comprises an organic polymer and wherein the organic polymer is a carboxylated polymer.

10. A detergent composition according to claim 1 wherein the detergent composition is in the form of a unit dose product.

11. A detergent composition according to claim 10 wherein the unit dose product comprises at least two compartments, wherein one compartment contains a composition in solid form and the other compartment contains a composition in liquid form.

12. A detergent composition according to claim 1 wherein the detergent composition is encompassed in an automatic dishwashing dosing element for use in an auto-dosing device.


SEQUENCE LISTING (4 sequences)

SEQ ID NO: 1 - protein, 269 aa, Bacillus Alkalophilus PB92
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 485 aa, Bacillus AA560
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 4 - protein, 485 aa, Bacillus sp. 707
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK